(19) United States
(12) Patent Application Publication
(10) Pub. No.: US 2023/0125402 A1
(43) Pub. Date: Apr. 27, 2023

(54) SYSTEMS AND METHODS FOR PRODUCING MIXTURES

(71) Applicants: Boston Scientific Medical Device Limited, Galway (IE); Boston Scientific Scimed, Inc., Maple Grove, MN (US)

(72) Inventors: Benjamin CLEVELAND, Bellingham, MA (US); Nitesh Ghananil BAVISKAR, Kalyan West (IN); Junaid Mohammed SHAIKH, Surat (IN); Richard Earl GRAFFAM, Pelham, NH (US); Joseph HERNANDEZ, Rutland, MA (US); Christopher WATSON, Lincoln, MA (US); Jennie CREEGAN, Minneapolis, MN (US); Kolbein KOLSTE, Acton, MA (US)

(73) Assignees: Boston Scientific Medical Device Limited, Galway (IE); Boston Scientific Scimed, Inc., Maple Grove, MN (US)

(21) Appl. No.: 17/970,705

(22) Filed: Oct. 21, 2022

Related U.S. Application Data
(60) Provisional application No. 63/262,948, filed on Oct. 22, 2021.

(51) Int. Cl. A61J 1/20

(52) U.S. Cl. CPC A61J 1/2058; A61J 1/201; A61J 1/202

(57) ABSTRACT

A system is disclosed for producing a mixture to deliver to a treatment site. The system can include a first chamber with an inner cavity including a first constituent and an outer cavity concentric with the first cavity and including a second constituent. A partition can be openable and separate the first and second cavities. A second chamber can include a third constituent and be concentric with the first chamber. Moving the first chamber and/or the second chamber relative to the other can cause the partition to open so that the first constituent and the second constituent mix with each other to form a first mixture. Applying a force to the system can cause the first mixture and the third constituent to egress through distal portions of the first and second chambers and mix together to form the mixture for delivery to the treatment site.

### CROSS-REFERENCE TO RELATED APPLICATIONS

This patent application claims the benefit of priority under 35 U.S.C. § 119 to U.S. Provisional Patent Application No. 63/262,948, filed Oct. 22, 2021, which is herein incorporated by reference in its entirety.

### FIELD

The present disclosure relates generally to compositions for injection to a patient, methods of preparation and use thereof, and devices comprising such compositions.

### BACKGROUND

Numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Since the conception of conformal radiotherapy, physicians have paid attention to the delivered dose to the target and surrounding tissues. Investigators have been able to correlate side effects to the amount of tissue receiving a certain radiation dose. And yet, time, distance, and shielding affect the dose that is delivered. The less time an area is exposed to radiation, the less dose delivered. The greater the distance from the radiation, the less dose delivered.

Current systems provide filler material to treatment sites to decrease the radiation dose to the rectum during radiotherapy for prostate cancer. However, the system that mixes the filler material in vitro includes numerous subcomponents, is complex to assemble, and rife with filler mixing errors prior to delivery within a patient at a treatment site. During the foregoing procedures, such errors and mishaps lead unnecessarily to patient risk, increased procedure time, and increased procedure costs. The solution of this disclosure resolves these and other issues of the art.

### SUMMARY

In accordance with certain aspects of the present disclosure, a system is disclosed for producing a mixture to deliver to a treatment site. The system can include a first chamber with an inner cavity including a first constituent and an outer cavity concentric with the first cavity and including a second constituent. A partition can be openable and separate the first and second cavities. A second chamber can include a third constituent and be concentric with the first chamber. Moving the first chamber and/or the second chamber relative to the other can cause the partition to open so that the first constituent and the second constituent mix with each other to form a first mixture. Applying a force to the system can cause the first mixture and the third constituent to egress through distal portions of the first and second chambers and mix together to form the mixture for delivery to the treatment site.

In accordance with certain aspects of the present disclosure, distal ends of the second cavity and a delivery lumen for the first mixture in fluid communication with the inner cavity are aligned with each other.

In accordance with certain aspects of the present disclosure, a pathway of the partition is opened by applying a torque to a cap of the outer cavity to compensate for a restoring force of a spring coupled to the outer cavity.

In accordance with certain aspects of the present disclosure, when the restoring force or a pressure of the outer cavity is higher on an outlet side of the partition or an inlet side of the partition has insufficient pressure, the partition remains closed.

In accordance with certain aspects of the present disclosure, a distal portion of the first and second chambers being joined in a central lumen, and wherein the first mixture and the third constituent mix in the central lumen to form the mixture.

In accordance with certain aspects of the present disclosure, one or more arms can be positioned on an external surface of at least one of the first and second chambers. The one or more arms are configured to facilitate the applying of the force to the system to cause the first mixture and the third constituent to egress through distally positioned ports and mix together within the central lumen to form the mixture.

In accordance with certain aspects of the present disclosure, the first constituent is a hydrophilic polymer, the second constituent is a diluent, and the first mixture is a precursor solution.

In accordance with certain aspects of the present disclosure, the first constituent is a diluent, the second constituent is a hydrophilic polymer, and the first mixture is a precursor solution.

In accordance with certain aspects of the present disclosure, the partition is positioned at a proximal end of the system in a first state then the partition is opened by being moved distally a first distance from the proximal end.

In accordance with certain aspects of the present disclosure, a bias element can be connected to a distal end of the first chamber and a proximal end of the second chamber. A distal end of the inner cavity can be connected to a plunger in the second chamber. The bias element can be configured to control a plunger of the first chamber so that moving the first chamber and/or the second chamber relative to the other causes the bias element to extend a distance between a distal end of the first chamber and a plunger of the second chamber.

In accordance with certain aspects of the present disclosure, moving the first chamber and/or the second chamber relative to the other causes the first chamber to telescope away from the second chamber while remaining at least partially concentric with each other and in fluid communication.

In accordance with certain aspects of the present disclosure, a distal end of the inner cavity is connected to a plunger in the second chamber.

In accordance with certain aspects of the present disclosure, a distal portion of the first cavity includes a delivery lumen in fluid communication with the inner cavity and extended within and concentric to the second cavity.

In accordance with certain aspects of the present disclosure, a needle can be connected to a distal end of the first and second cavities.

In accordance with certain aspects of the present disclosure, a distal portion of the first and second chambers can be joined in a central lumen positioned in a connector removably connected to a distal end of the first and second chambers. The connector can include a first tube configured to be in fluid communication with a first port at the distal end of the first chamber and the central lumen of the connector. The connector can include a second tube configured to be in fluid communication with a second port of the second chamber and the central lumen of the connector.

In accordance with certain aspects of the present disclosure, a method is disclosed for producing a mixture with a mixing system to deliver to a treatment site. The mixing system can include a first chamber including an inner cavity including a first constituent and an outer cavity concentric with the first cavity and including a second constituent. A partition can separate the first and second cavities. A second chamber can include a third constituent. The second chamber can be concentric with the first chamber. The method can include moving the first chamber and/or the second chamber relative to the other causing the partition to open so that the second constituent moves from the outer cavity through the partition and mixes with the first constituent in the inner cavity to form a first mixture; and applying a force to the system causing the first mixture and the third constituent to egress through distal portions of the first and second chambers and mix together to form the mixture for delivery to the treatment site.

In accordance with certain aspects of the present disclosure, the step of moving the first chamber and/or the second chamber relative to the other includes opening the partition by applying a torque to a cap of the outer cavity to compensate for a restoring force of a spring coupled to the outer cavity.

In accordance with certain aspects of the present disclosure, the method can include maintaining the partition closed when the restoring force or a pressure of the outer cavity is higher on an outlet side of the partition.

In accordance with certain aspects of the present disclosure, the method can include maintaining the partition closed when an inlet side of the partition has insufficient pressure.

In accordance with certain aspects of the present disclosure, the method can include controlling, by a bias element coupled to a distal end of the first chamber, a plunger of the first chamber so that moving the first chamber and/or the second chamber relative to the other causes the bias element to extend a distance between a distal end of the first chamber and a plunger of the second chamber.

In accordance with certain aspects of the present disclosure, the step of moving the first chamber and/or the second chamber relative to the other causes the first chamber to telescope away from the second chamber while remaining at least partially concentric with each other and in fluid communication.

In accordance with certain aspects of the present disclosure, the method can include connecting a proximal end of the inner cavity to a plunger in the second chamber.

In accordance with certain aspects of the present disclosure, the method can include positioning one or more arms on an external surface of at least one of the first and second chambers, the one or more arms configured to facilitate applying the force to the system to cause the first mixture and the third constituent to egress through distally positioned ports and mix together to form the mixture.

In accordance with certain aspects of the present disclosure, distal ends of the second cavity and a delivery lumen for the first mixture in fluid communication with the inner cavity are aligned with each other.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIGS. 1A-1B depict the prostate, rectum, and Denonvilliers' space between the prostate and rectum.

FIGS. 2A and 2B depict perspective views of an exemplary mixing system in accordance with certain aspects of the present disclosure.

FIG. 3A depicts a side cross-section view of an exemplary mixing system, in accordance with certain aspects of the present disclosure.

FIG. 3B depicts a close-up, side cross-section view of an example step in a method using an example mixing system, in accordance with certain aspects of the present disclosure.

FIG. 4A depicts an example step in a method using an example mixing system, in accordance with certain aspects of the present disclosure.

FIG. 4B depicts an example step in a method using an example mixing system, in accordance with certain aspects of the present disclosure.

FIG. 5 depicts an example step in a method using an example mixing system, in accordance with certain aspects of the present disclosure.

FIGS. 6A-6C depict example steps in a method of priming an example connector, in accordance with certain aspects of the present disclosure.

FIG. 7 depicts a flow diagram of a method of using a mixing system according to certain aspects of this disclosure.

### DETAILED DESCRIPTION

Particular aspects of the present disclosure are described in greater detail below. The terms and definitions provided herein control, if in conflict with terms and/or definitions incorporated by reference.

Particular aspects of the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise.

As used herein, “approximately” and “about” refer to being nearly the same as a referenced number or value. As used herein, the terms “approximately” and “about” should be understood to encompass ±10% of a specified amount or value (e.g., “about 90%” can refer to the range of values from 81% to 99%).

As used herein, “operator” can include a doctor, surgeon, or any other individual or delivery instrumentation associated with delivery or use of a mixing system as such systems are described throughout this disclosure.

The compositions herein may be used in various medical procedures, including but not limited to injected to create additional space between the rectum and prostate during treatment, for example in the Denonvilliers' space, thereby reducing rectal radiation dose and associated side effects. Certain embodiments of the disclosure include placing a filler between the radiation target tissue and other tissues. The filler can be a gel composition that increases the distance between the target tissue and other tissues so that the other tissues receive less radiation.

It is understood that “Denonvilliers' space” is a region located between the rectum and prostate. Certain embodiments provide a method of displacing a tissue to protect the tissue against the effects of a treatment involving radiation or cryotherapy. One embodiment involves using a filler mixed by a mixing system of this disclosure to displace the tissue relative to a tissue that is to receive the treatment. Another embodiment involves introducing a filler mixed by a mixing system of this disclosure to displace a first tissue and radiating a second tissue, particularly a second tissue that is close to the first tissue. In another embodiment, the method includes the steps of injecting a filler into a space between tissues; and may further include irradiating one of the tissues so that the other tissue receives less radiation than it would have in the absence of the filler.

Certain embodiments also provide methods for treating a tissue of a body by radiation. In one embodiment, the method includes the steps of injecting an effective amount of a filler into a space between a first tissue (e.g., prostate) of a body and a second tissue (e.g., rectum), which can be a critically sensitive organ; and treating the first tissue by radiation whereby the filler within the space reduces passage of radiation into the second tissue. Tissue is a broad term that encompasses a portion of a body: for example, a group of cells, a group of cells and interstitial matter, an organ, a portion of an organ, or an anatomical portion of a body, e.g., a rectum, ovary, prostate, nerve, cartilage, bone, brain, or portion thereof.

The gel of the filler can include polymeric materials which are capable of forming a hydrogel may be utilized. In one embodiment, the polymer forms a hydrogel within the body. A hydrogel is defined as a substance formed when an organic polymer (natural or synthetic) is cross-linked via covalent, ionic, or hydrogen bonds to create a three-dimensional open-lattice structure which entraps water molecules to a gel. Naturally occurring and synthetic hydrogel forming polymers, polymer mixtures, and copolymers may be utilized as hydrogel precursors.

In some aspects, the hydrogel can be formed by a composition formed by constituents (e.g., mixing accelerant fluid, diluent, and PEG together) and may include one or more polysaccharide compounds or a salt thereof. For example, the composition may include a cellulose compound such as carboxymethyl cellulose (CMC) or salt thereof (e.g., CMC) sodium, xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be cross-linked with a suitable crosslinking compound, such as butanediol diglycidyl ether (BDDE). In some aspects, the polysaccharide may be a homopolysaccharide or a heteropolysaccharide

The present disclosure also provides mixing systems to form the gel composition and corresponding medical devices for use and/or delivery to a treatment site of a patient. According to some aspects of the present disclosure, the mixing system may include a plurality of reservoirs with respective lumens. Collectively, the lumens therein may serve as a container for constituents to mix the gel composition of this disclosure. Suitable reservoirs may include, for example, syringes (e.g., a syringe barrel compatible with a manual or automatic injection system) and other fluid containers configured for use with a suitable injection needle. Exemplary materials suitable for the reservoir include, but are not limited to, cyclic olefin polymer, polypropylene, polycarbonate, polyvinyl chloride, and glass. In some aspects, one of these materials (e.g., cyclic olefin copolymer specifically) can have a coating applied to it, such as SiO₂), which is advantageous so the coating can perform as a primary oxygen barrier, behave as a glass-like layer, and can be applied using a vapor deposition process.

According to some aspects of the present disclosure, the compositions may include at least one accelerant (e.g., an activating agent) combined with a precursor mixed from a diluent (e.g., mostly water) and polyethylene glycol (PEG). In some examples, the composition may be or include a gel with a desired gel strength and/or viscosity, such as a biocompatible gel suitable for injection (e.g., through a needle).

The hydrophilic polymer can be any gelling agent(s), including natural ones or synthetic in origin, and may be anionic, cationic, or neutral. Non-limiting examples of the gelling agents include polysaccharides such as gellan gum, xanthan gum, gum arabic, guar gum, locust bean gum, alginate, and carrageenans.

The concentrations of gelling agent(s) in the composition described in this disclosure may range from about 0.01% to about 2.0% by weight with respect to the total weight of the composition, such as from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, the composition may have a viscosity ranging from about 0.001 Pascal-second (Pa·s) to about 0.100 Pa·s at a shear rate of 130 s⁻¹, such as, e.g., from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, the composition may have a viscosity ranging from about 0.001 Pa·s to about 0.050 Pa·s at a shear rate of 768 s⁻¹, such as, e.g., from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The mixing system herein may include or be removably connected to one or more needles. In some examples, the needle may be a hypodermic needle, and may range from a size of 7 gauge (4.57 mm outer diameter (OD), 3.81 mm inner diameter (ID)) to 33-gauge (0.18 mm OD, 0.08 mm ID), e.g., a size of 16 gauge (1.65 mm OD, 1.19 mm ID), 18 gauge, 21 gauge (0.82 mm OD, 0.51 mm ID), 22 gauge (0.72 mm OD, 0.41 mm ID), 23 gauge (0.64 mm OD, 0.33 ID), or 24 gauge (0.57 mm OD, 0.31 mm ID). Exemplary materials for the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

According to some aspects of the present disclosure, the filler compositions herein, e.g., the compositions prepared by the methods herein may have sufficient strength, e.g., gel strength, to withstand the forces and thus minimizing the effects of the forces on the continuity of the three-dimensional gel network. In the meantime, the composition with sufficient strength may have a viscosity suitable for injection, e.g., a viscosity that does not render the composition stuck in the reservoir(s), delivery lumen, or a needle connected therewith.

According to some aspects of the present disclosure, the composition may maintain its three-dimensional structure until the gel is injected through a needle, whereupon the structure may form fragments of the original continuous, three-dimensional network. Those gel fragments may have a diameter corresponding to the diameter of the injection needle, such that the fragments are as large as possible in-vivo to retain as much of the three-dimensional structure of the gel as possible. Injection of these larger-sized particles or fragments is believed to increase the amount of time the gel remains within the tissue.

The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lbf to about 25 lbf, such as from about 10 lbf to about 20 lbf, e.g., about 15 lbf. The loads measured for a given gel concentration may vary for different needles and flow rates.

According to some aspects of the present disclosure, the size of the needle may be chosen based on the viscosity and/or components of the composition, or vice versa. According to some aspects of the present disclosure, the size of the needle may be 23 gauge or 25 gauge. In some cases, a larger size of 18 gauge, 20 gauge, 21 gauge, or 22 gauge may be used to inject the compositions herein.

According to some aspects of the present disclosure, the mixing system of this disclosure can be included in a kit for introducing a filler into a patient, whereby the filler can include any of the gel compositions of this disclosure. Kits or systems for mixing a gel composition of this disclosure, such as hydrogels, may be prepared so that the precursor(s) and any related activating agent(s) are stored in the kit with diluents as may be needed. Applicators may be used in combination with the same. The kits can be manufactured using medically acceptable conditions and contain components that have sterility, purity and preparation that is pharmaceutically acceptable. Solvents/solutions may be provided in the kit or separately. The kit may include syringes and/or needles for mixing and/or delivery. The kit or system may comprise components set forth herein.

During some examples of use, once saline has been injected to the treatment site, a mixing system can be connected to a needle (e.g., an 18 gauge spinal needle) to then inject a 5-10 mm layer of filler (e.g., gel composition) along the posterior wall of the prostate between the prostate and rectum. Once the filler has been injected into the space between the rectum and prostate, ultrasound images can be obtained.

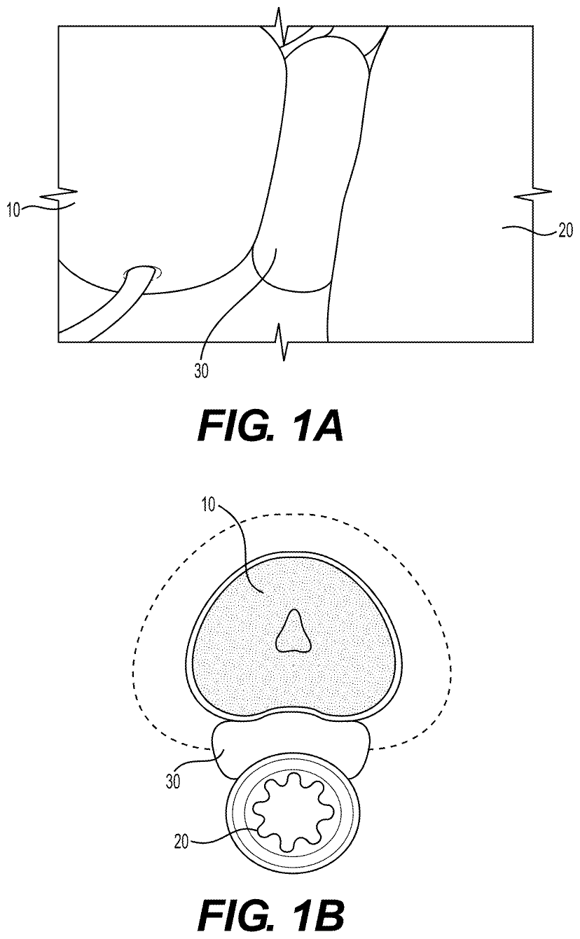

Turning to the drawings, FIG. 1A is a perspective view and FIG. 1B is a partial cross-section view illustrating example filler 30, in the form of a gel composition having been delivered by the mixing system of this disclosure between rectum 20 and prostate 10 of a patient in Denonvilliers' space.

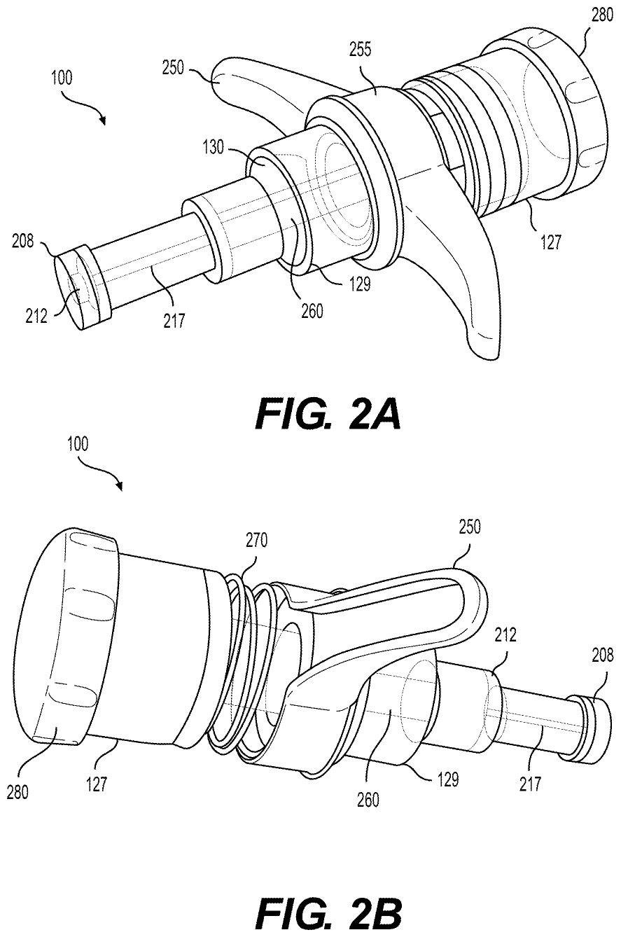

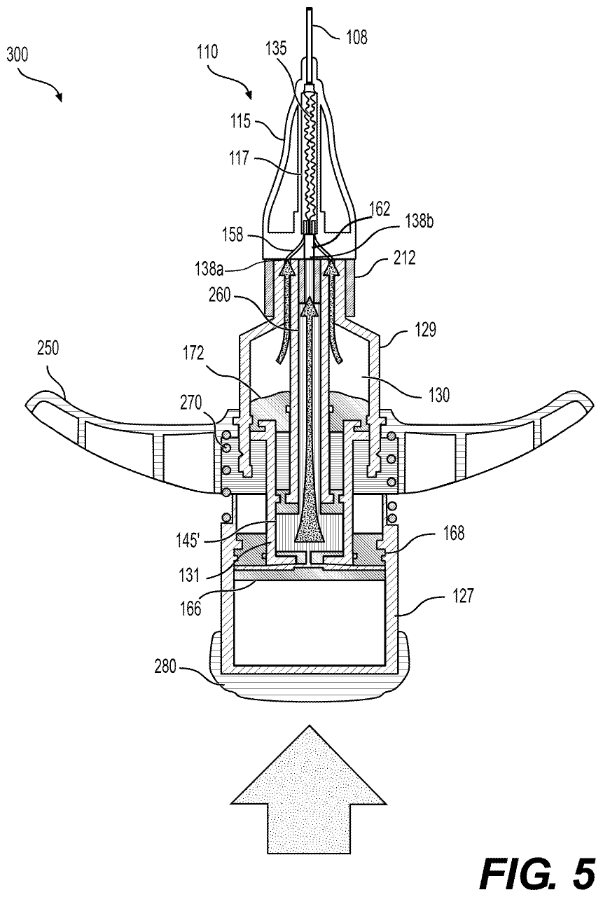

FIG. 2A shows an upper perspective view of an exemplary mixing system 100 in accordance with certain aspects of the present disclosure for mixing a gel composition for use as filler 30. FIG. 2B shows a lower perspective view of system 100. The system 100 can be packaged in a kit and be attachable to a needle 108 or a needle assembly 110 (which can include needle 108) attachable to system 100, as in FIG. 5. Needle 108 can be any needle of this disclosure suitable for hydrodissection as well as delivering a mixture, such as the gel composition (e.g., filler 30) to the treatment site.

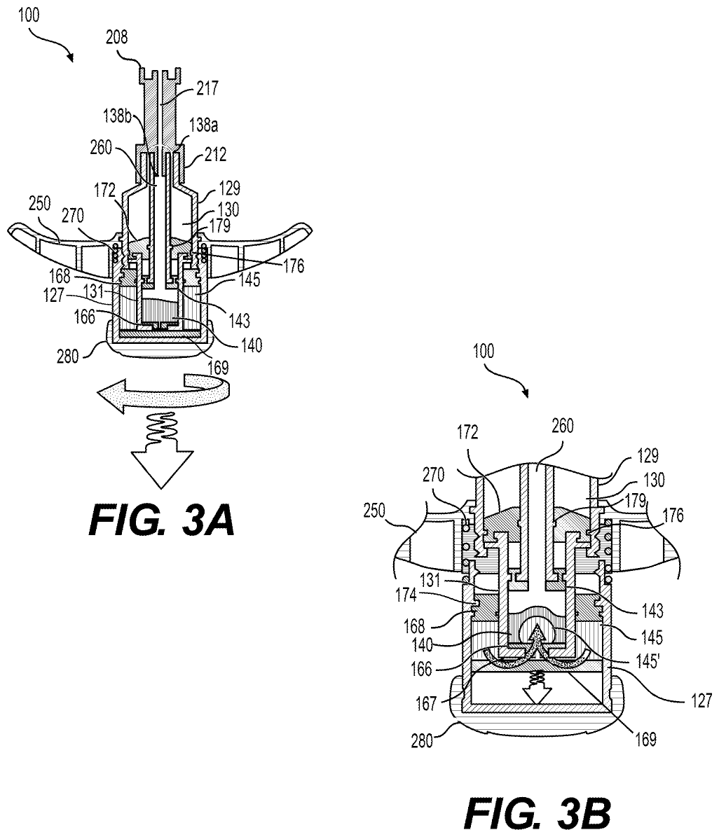

Turning to FIG. 3A, system 100 is shown with a first chamber 127 with an inner cavity 131 including constituent 140 (e.g., an activating agent such as a hydrophilic polymer, PEG, etc.) or some other constituent, such as constituent 145, for producing a precursor for a gel composition mixture of filler 30. As used herein, the term “fluid” is defined broadly and can include liquids, gels and particulate matter such as granules, pellets, or powders capable of flowing between locations, or any combination of liquids, gels, oils, and/or particulate matter (e.g., granules, pellets, or powders). The diluent used in systems of this disclosure, including system 100, can be a branched polymer having a plurality of succinimidyl termini dissolved in a low pH (4.0) containing a low molecular weight precursor comprising nucleophiles, though other diluent fluid solutions are contemplated within the scope of this disclosure. Chamber 127 can include a closed bottom end rotatably connected to cap 280. An outer cavity can be defined between walls of cavity 131 and chamber 127. Cavity 131 can be a separate component from chamber 127 and be concentric with chamber 127. Cavity 131 can include constituent 145 (e.g., diluent, which can be the other of constituent 140 to mix and form precursor 145′).

Second chamber 129 can be positioned at least partially distal of and concentric with chamber 127. Chamber 129 can include a substance (e.g., accelerant). An outer diameter of chamber 129 can be configured to engage with a distal end of an inner diameter of chamber 127. For example, an inner surface of the distal end of chamber 127 can threadingly engage with an outer surface of a proximal end of chamber 129. However, chambers 127, 129 can be movably coupled together in other approaches (e.g., latch, groove, guide, or other manners of adjustably coupling two chambers). Chamber 129 can terminate in one or more ports 138a at its distal end. Lumen 260 similarly can terminate in a port 138b at its distal end, which can be aligned with port 138a. In some aspects, lumen 260 can be concentric with and run through chamber 129. Needle adaptor 212 can be coupled to a distal end of chamber 129 and lumen 260. Adaptor 212 can include fluid ports to receive fluids through ports 138a, 138b. Adaptor 212 can include a central lumen 217, which in some examples can include a static mixer. Fluids from chamber 129 and lumen 260 can mix within lumen 217 to form the gel composition of filler 30 and ultimately be delivered through a needle 108 connected to needle coupler 208.

A plunger stopper 168 can be coupled to an inward protrusion 174 of chamber 127. In some examples, protrusion 174 can be extended circumferentially within an inner surface of chamber 127 to couple with a corresponding inward notch of stopper 168. In some examples, protrusion 174 can be positioned between the proximal and distal ends of chamber 127 can be flush or in contact with a proximal end of chamber 129. A proximal end of spring 270 can control a distal end of chamber 127 and be mechanically linked to an outer surface of the distal end of chamber 127. Stopper 168 can be positioned adjacent to a proximal end of spring 270 but on an inner surface of chamber 127.

A proximal end of chamber 129 can include a plunger stopper 172. Stopper 172 can be mechanically linked to an outer, distal end of cavity 131 and an inward protrusion 176 of a proximal end of chamber 129. In some examples, protrusion 176 can be extended circumferentially within an inner surface of chamber 127 to couple with a corresponding inward notch of stopper 168. A central lumen 260 can run through and be axially aligned with chambers 127, 129. Lumen 260 can include a protrusion 179 extended outward radially and circumferentially about lumen 260 to couple with stopper 172. Distal thereof at a distal end of lumen 260 can be a lower, proximal plunger 143 through which fluids can flow from cavity 131.

One or more arms 250 can provide a central interface through which chambers 127, 129 can engage to telescope with respect to each other. In some aspects, a distal end of spring 270 can be positioned anchored, adjacent therewith on an outer surface of chamber 129 and the interface of one or more arms 250. For example, chamber 129 can include an outward, radial protrusion configured to couple with a corresponding notch of one or more arms 250. The one or more arms 250 can also include a notch or receiving surface in which a distal end of spring 270 can be mechanically attached or otherwise anchored. While spring 270 is shown in this disclosure, other bias elements are contemplated, including but not limited to one or more coils, resistance bands, or elastomers to provide restorative force.

A proximal end of spring 270 can control a distal end of chamber 127 and be mechanically linked to an outer surface of the distal end of chamber 127. Stopper 168 can be positioned adjacent the proximal end of spring 270 but on an inner surface of chamber 127. In some aspects, as seen in FIGS. 3A-3B, the one or more arms 250 can articulate outward so that a user can grasp thereon while moving, rotating, or otherwise applying a force to cap 280. Spring 270 can be positioned between a distal, open end of chamber 127 and a proximal, open end of chamber 129.

Cap 280 can be mechanically linked to spring 270 whereby spring 270 can be configured to apply a restoring force applied to chamber 127 to draw chamber 127 towards chamber 129, as in the idle state of FIG. 3A. An openable partition 166, which can be an openable valve, can separate a proximal end of cavity 131 and constituent 145 of chamber 127. Partition 166 can include an openable seal or membrane coupled with a lower disc 169 coupled to a proximal end of cavity 131. In some aspects, partition 166 can be initially positioned at or adjacent a proximal end of cavity 131 sealed against disc 169. In some aspects, applying a force to cap 280 (e.g., rotating cap 280) causes spring 270 to elongate, as in FIG. 3B. In turn, chamber 127 can move away from chamber 129 while disc 169 and cavity 131 remain relatively stationary. Moving chamber 127 away from chamber 129 can cause a distance between stopper 168 and disc 169 to decrease and cause one or more fluid pathways 167 of partition 166 to open (e.g., when a predetermined pressure is achieved). In some aspects, to open pathway(s) 167 of partition 166, cap 280 can have an applied torque in order to compensate for the restoring force of spring 270. When the restoring force by spring 270 or pressure is higher on the outlet side of partition 166 in cavity 131 or the inlet side of partition 166 does not have sufficient pressure, pathway(s) 167 of partition 166 in some aspects can remain closed.

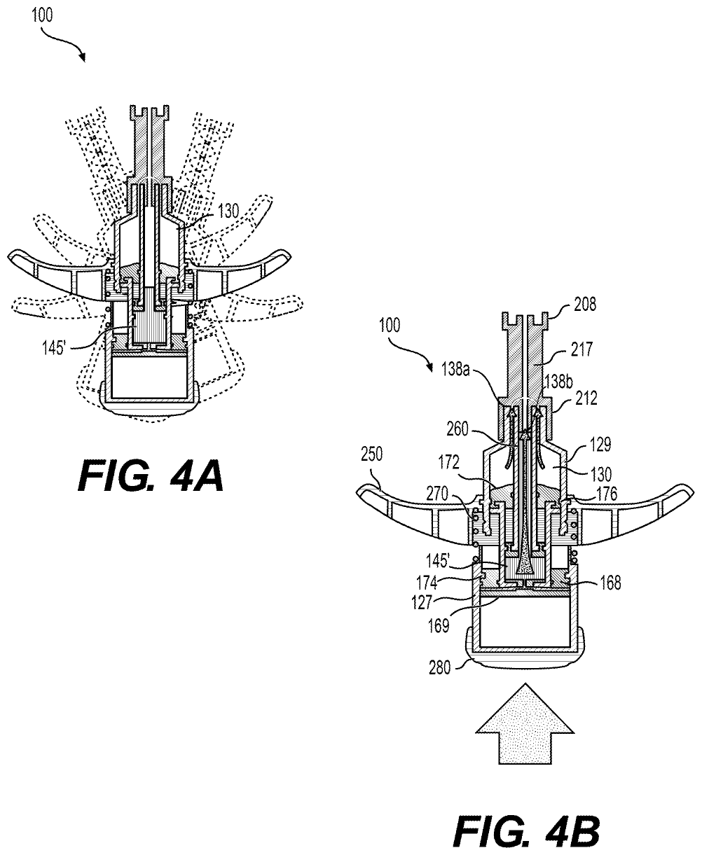

In some aspects, elongating spring 270 can increase a distance between a distal end of chamber 127 and stopper 172 of chamber 129 and open partition 166. This is more clearly seen in FIGS. 3A-3B where flow enters the input port of partition 166 when it has enough force by cap 280 being moved or otherwise rotated to overcome the spring force imparted by spring 270. Once overcome, disc 169 is pushed thereby opening pathway 167 of partition 166 allowing constituent(s) to move through partition 166 and into cavity 131. When the input force by cap 280 is no longer high enough, or there is a backpressure, then the backpressure and/or spring 270 can push disc 169 against the orifice and shut partition 166. In some examples, once disc 169 and stopper 168 are aligned or in contact, as in FIGS. 4A and 4B, this can indicate that cavity 131 has all fluid from chamber 127.

Once the constituents 140, 145 are present in cavity 131, as shown moving from FIG. 3A (idle state) to FIG. 3B (fluid pathway(s) 167 open), system 100 can be shaken as in FIG. 4A thereby forming precursor 145′ in cavity 131. For example, system 100 can be shaken back and forth to ensure precursor 145′ forms as a result of mixing between constituents 140, 145. While shaking occurs, a constituent 130 (e.g., an accelerant) remains in chamber 129. Constituent 130 can be an accelerant that is mixable with precursor 145′ to form the mixture of filler 30 (e.g., a gel composition). Preferably, the shaking action of FIG. 4A is done while ports 138a, 138b are oriented generally upward. However, system 100 is not so limited and the shaking to effect proper mixing of precursor 145′ can be performed in other orientations (e.g., generally downward, etc.), as needed or required. While certain steps are shown as a sequence between each of FIGS. 3A-4A, in other embodiments, fewer steps are contemplated and the order by which steps are performed can be different than what is illustrated.

With precursor 145′ formed and present in lumen 260 and constituent 130 in chamber 129, a needle 108 can be attached to coupler 208 and a force can be applied to cap 280, as denoted by the upward arrow in FIG. 4B. In some examples, mixing of fluids in lumen 217 or otherwise causing fluids to egress from lumen 260 and chamber 129 can be done by applying a force system 100 (e.g., moving one or more arms 250 towards cap 280 while applying an opposite force on cap 280). A user can compress system 100 in one simple movement advantageously by using digits on the one or more arms and cap 280. In so doing, precursor 145′ can egress through port 138b while constituent 130 can egress through port 138a to mix within lumen 217. While not shown, it is contemplated that any unwanted air in system 100 can also be purged in this step through respective ports 138a, 138b or lumen 217 prior to connecting needle 108.

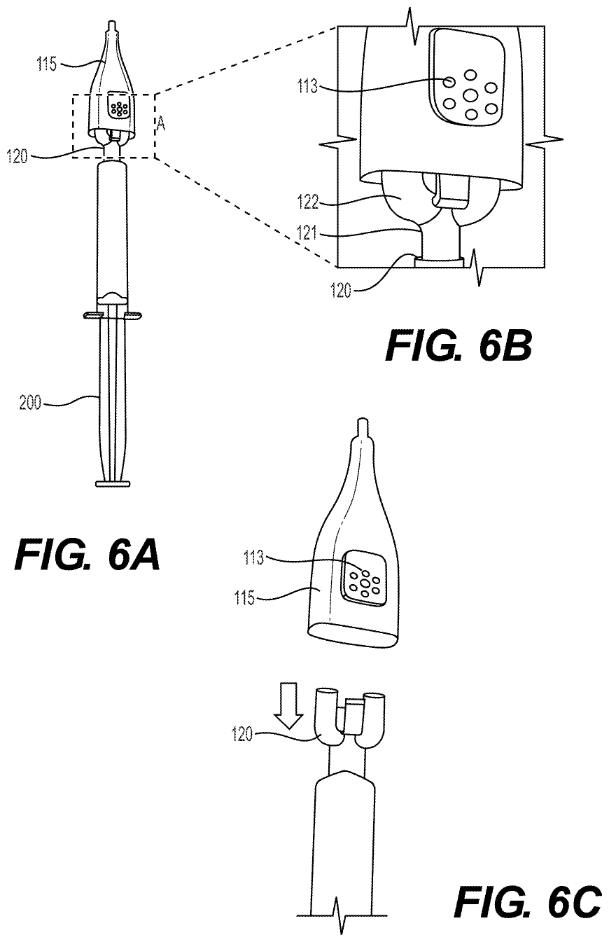

FIG. 5 shows a perspective view of another exemplary mixing system 300 in accordance with certain aspects of the present disclosure for mixing a gel composition for use as filler 30. The system 300 is substantially similar to system 100, except that instead of couple 208 directly attaching to needle 108, a needle assembly 110 is positioned connected to end 212 and in fluid communication with ports 138a, 138b. Assembly 110 is advantageous as it can facilitate connection with needle 108 as well as mixing of precursor 145′ and constituent 130 to form the mixture of filler 30. Assembly 110 can include needle 108, which can be connected to a distal end of a primed connector 115. Aspects of priming connector 115 are discussed more in FIGS. 6A-6C. Connector 115 can be relatively hollow with a tapered or Y-shape profile for its outer surface. A central lumen 117 can run through connector 115 whereby each of ports 138a, 138b can be in fluid communication with a proximal end of lumen 117 of connector 115. Lumen 117 can include a static mixer 135 so that fluid from respective chambers 127, 129 can mix together and form the gel composition to be delivered through needle 108.

Separately, in FIG. 6A, connector 115 is shown connected to syringe 200 via adaptor 120, as illustrated more clearly in FIG. 6B which is a close-up of section A. Adaptor 120 can provide a fluid bridge 122 from each of tubes 158, 162 (e.g., hypotubes) of connector 115 to syringe 200. Connector 115 can include an externally positioned button 113 to open and close corresponding connecting latches of connector 115. Adaptor 120 can couple to syringe 200 with a luer fitting (e.g., end 121) or any other connector operable to connect with a distal end of syringe 200. While not shown, during use connector 115 is contemplated to be connected to needle 108 for hydrodissection at the treatment site with saline from the syringe 200. After hydrodissection with syringe 200, adaptor 120 can be released from connector 115, as shown in FIG. 6C. However, other coupling approaches between connector 115, adaptor 120, and syringe 200 are contemplated as needed or required. For example and without limitation, snap fit connectors, magnetic connectors, female-male connectors, hook and loop fasteners and the like are contemplated.

In some aspects, tubes 158, 162 can pierce a corresponding membrane or seal of ports 138a, 138b. In this respect, once precursors 145′ is in position in cavity 131 and constituent 130 is positioned in chamber 129 and connector 115 assembled thereto, precursor 145′ and constituent 130 can be caused to egress through respective ports 138a, 138b and respective tubes 158, 162 to mix with each other in lumen 117. Tubes 158, 162 can form a Y-shape as shown, though any other shape can be used as needed or required.

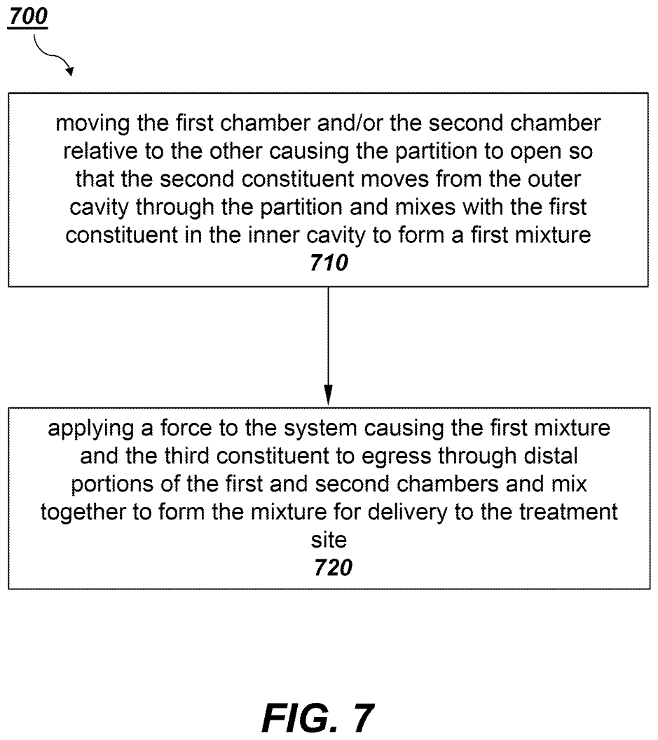

FIG. 7 depicts a method 700 of using any of the herein disclosed mixing systems. Step 710 of method 700 can include moving the first chamber and/or the second chamber relative to the other causing the partition to open so that the second constituent moves from the outer cavity through the partition and mixes with the first constituent in the inner cavity to form a first mixture. Step 720 of method 700 can include applying a force to the system causing the first mixture and the third constituent to egress through distal portions of the first and second chambers and mix together to form the mixture for delivery to the treatment site. Method 700 can end after step 720. In other embodiments, additional or fewer steps according to the examples described above can be performed.

The systems and methods of this disclosure are beneficial by reducing the number of system components, are relatively simply to assemble and operate, with minimal mixing errors prior to delivery within a patient at a treatment site. Other aspects and embodiments of the present disclosure will be apparent to those skilled in the art from consideration of the specification and practice of the embodiments disclosed herein.

While certain features of the present disclosure are discussed within the context of exemplary procedures, the compositions, systems, and methods may be used for other medical procedures according to the general principles disclosed. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein. It is intended that the specification and examples be considered as exemplary only, with a true scope and spirit of the present disclosure being indicated by the following claims.

What is claimed is:

1. A system for producing a mixture to deliver to a treatment site, comprising:
a first chamber comprising an inner cavity comprising a first constituent and an outer cavity concentric with the first cavity and comprising a second constituent;
a partition being openable and separating the first and second cavities;
a second chamber comprising a third constituent, the second chamber being concentric with the first chamber;
wherein moving the first chamber and/or the second chamber relative to the other causes the partition to open so that the first constituent and the second constituent mix with each other to form a first mixture; and
wherein applying a force to the system causes the first mixture and the third constituent to egress through distal portions of the first and second chambers and mix together to form the mixture for delivery to the treatment site.

2. The system of claim 1, wherein distal ends of the second cavity and a delivery lumen for the first mixture in fluid communication with the inner cavity are aligned with each other.

3. The system of claim 1, wherein a pathway of the partition is opened by applying a torque to a cap of the outer cavity to compensate for a restoring force of a spring coupled to the outer cavity.

4. The system of claim 3, wherein when the restoring force or a pressure of the outer cavity is higher on an outlet side of the partition or an inlet side of the partition has insufficient pressure, the partition remains closed.

5. The system of claim 1, wherein a distal portion of the first and second chambers being joined in a central lumen, and wherein the first mixture and the third constituent mix in the central lumen to form the mixture.

6. The system of claim 5, further comprising:
one or more arms positioned on an external surface of at least one of the first and second chambers, the one or more arms configured to facilitate the applying of the force to the system to cause the first mixture and the third constituent to egress through distally positioned ports and mix together within the central lumen to form the mixture.

7. The system of claim 1, wherein the first constituent is a hydrophilic polymer, the second constituent is a diluent, and the first mixture is a precursor solution.

8. The system of claim 1, wherein the first constituent is a diluent, the second constituent is a hydrophilic polymer, and the first mixture is a precursor solution.

9. The system of claim 1, wherein moving the first chamber and/or the second chamber relative to the other causes the first chamber to telescope away from the second chamber while remaining at least partially concentric with each other and in fluid communication.

10. The system of claim 1, wherein a distal end of the inner cavity is connected to a plunger in the second chamber.

11. The system of claim 1, wherein a distal portion of the first cavity comprises a delivery lumen in fluid communication with the inner cavity and extended within and concentric to the second cavity.

12. A method for producing a mixture with a mixing system to deliver to a treatment site, the mixing system comprising a first chamber comprising an inner cavity comprising a first constituent and an outer cavity concentric with the first cavity and comprising a second constituent, a partition separating the first and second cavities, and a second chamber comprising a third constituent, wherein the second chamber is concentric with the first chamber, the method comprising:
moving the first chamber and/or the second chamber relative to the other causing the partition to open so that the second constituent moves from the outer cavity through the partition and mixes with the first constituent in the inner cavity to form a first mixture; and
applying a force to the system causing the first mixture and the third constituent to egress through distal portions of the first and second chambers and mix together to form the mixture for delivery to the treatment site.

13. The method of claim 12, wherein the step of moving the first chamber and/or the second chamber relative to the other comprises opening the partition by applying a torque to a cap of the outer cavity to compensate for a restoring force of a spring coupled to the outer cavity.

14. The method of claim 13, further comprising:
maintaining the partition closed when the restoring force or a pressure of the outer cavity is higher on an outlet side of the partition.

15. The method of claim 13, further comprising:
maintaining the partition closed when an inlet side of the partition has insufficient pressure.

16. The method of claim 12, further comprising:
controlling, by a bias element coupled to a distal end of the first chamber, a plunger of the first chamber so that moving the first chamber and/or the second chamber relative to the other causes the bias element to extend a distance between a distal end of the first chamber and a plunger of the second chamber.

17. The method of claim 12, wherein the step of moving the first chamber and/or the second chamber relative to the other causes the first chamber to telescope away from the second chamber while remaining at least partially concentric with each other and in fluid communication.

18. The method of claim 12, further comprising:
connecting a proximal end of the inner cavity to a plunger in the second chamber.

19. The method of claim 12, further comprising:
positioning one or more arms on an external surface of at least one of the first and second chambers, the one or more arms configured to facilitate applying the force to the system to cause the first mixture and the third constituent to egress through distally positioned ports and mix together to form the mixture.

20. The method of claim 12, wherein distal ends of the second cavity and a delivery lumen for the first mixture in fluid communication with the inner cavity are aligned with each other.
